(19) United States
(12) Patent Application Publication
(10) Pub. No.: US 2002/0072719 A1
(43) Pub. Date: Jun. 13, 2002

(54) Constant speed sample collection or injection system

(76) Inventors: Joel S. Douglas, Los Altos Hills, CA (US); Robert Hugo, Gilroy, CA (US); David M. Kelso, Wilmatte, IL (US)

Correspondence Address: T. Gene Dillahunty, BURNS. DOANE, SWECKER & MATHIS, L.L.P., P.O. Box 1404, Alexandria,, VA, 22313-1404, US

(21) Appl. No.: 09/734,760

(22) Filed: Dec. 13, 2000

Related U.S. Application Data
(60) Provisional application No. 60/170,569, filed on Dec. 13, 1999.

(51) Int. Cl. A61M 5/00

(52) U.S. Cl. 604/257

(57) ABSTRACT

A constant speed fluid sample collection system for use with a catheter, needle, or the like in communication with a patient's vein is useful to extract a blood sample. The system uses a constant force system to not disrupt the components of the fluid, such as platelets found in blood. The system includes a syringe device and moves a plunger at a constant velocity. The device includes a housing, a piston rod movably mounted within the housing, and a brake mechanism for controlling the movement of the piston with respect to a syringe positioned within the housing. The plunger retracts into the drive device as it is being withdrawn.

This application is related and claims priority under 35 U.S.C. § 119 and/or § 120 to U.S. application Ser. No. 60/170,569, “Constant Speed Sample Collection System”, filed Dec. 13, 1999, by Joel S. Douglas, the entire contents of which are incorporated by reference herein.

### BACKGROUND OF THE INVENTION

1. Field of the Invention

The present invention relates to a fluid sample collection device which extracts a sample of body fluid, such as blood, from a patient and uses a constant force or speed control to limit the disturbance of the sample to permit analysis of the sample.

2. Brief Description of the Related Art

Many medical procedures in use today require a relatively small sample of blood, in the range of 50-500 milliliters. It is typically more cost effective and less traumatic to the patient to obtain such a sample by lancing or piercing the skin at a selected location, using a catheter to open a wound in a vein, to enable the collection of blood. The phlebotomist can draw then venous blood into a device which is useable in the analysis mechanism and does not overly disturb the sample.

Blood collection tubes are commonly used by doctors, nurses and other persons to draw a sample of a body fluid from a patient or to receive a fluid sample from another vessel. Such tubes are ordinarily evacuated, and include a pierceable closure. During one typical use of a blood collection tube, one end of a double-ended needle assembly is used to pierce a vein. The evacuated blood collection tube is then urged towards the second end of the double-ended needle assembly until its closure is pierced. Blood is thereby drawn into the tube. The double-ended needle assembly is ordinarily mounted to a holder having a tubular body. The blood collection tube is inserted within the holder's tubular body in order to engage the second end of the double-ended needle assembly.

The maintenance of a vacuum within the blood collection tube is important for the proper operation of such devices. Blood collection tubes may be stored for considerable lengths of time before they are used, yet loss of vacuum during storage or at any time can render the collection tube less effective or useless. Furthermore, the vacuum is such collection tubes can cause activation of the platelets due to turbulence in the blood caused by the vacuum.

In institutional settings, it is often desirable to collect the sample from the patient and then introduce the sample to a test device in a controlled fashion. Some blood glucose monitoring systems, for example, require that the blood sample be applied to a test device which is in contact with a test instrument. In such situations, bringing the finger of a patient directly to the test device poses some risk of contamination from blood of a previous patient. With such systems, particularly in hospital settings, it is common to lance a patient, collect a sample in a micropipette via capillary action, and then deliver the sample from the pipette to the test device.

### SUMMARY OF THE INVENTION

According to a first exemplary embodiment, a fluid sample collection device comprises a housing having a hollow interior, a plunger positioned at least partially within said housing interior and movable within said housing, a piston attached to the plunger and extending from the housing, a spring positioned to move the plunger in a first direction, a wall surrounding and engaging a portion of the plunger to generate a friction force when the plunger moves relative to the wall, and a brake mechanism in the plunger which adjusts the friction force.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to preferred embodiments of the apparatus and method, given only by way of example, and with reference to the accompanying drawings, in which:

FIG. 1 illustrates an exploded perspective view of an exemplary embodiment in accordance with the present invention;

FIG. 2 illustrates a longitudinal cross-sectional view of the device illustrated in FIG. 1, with the device oriented to withdraw a sample of a fluid;

FIG. 3 illustrates a view similar to that illustrated in FIG. 2, with the device oriented after having withdrawn a sample of fluid; and

FIG. 4 illustrates an enlarged cross-sectional view, with portions broken away, of interior portions of the device illustrated in FIGS. 1-3 in two different orientations.

### DESCRIPTION OF THE PREFERRED EMBODIMENTS

In general, the present invention assists in ensuring that a sufficiently large sample of body fluid or blood can be drawn and used in an analysis machine without having to be transferred to another device. The sample is not disturbed by excess turbulence, as in prior devices, so that the sample can be properly analyzed. By controlling the speed of the collection of the body fluid, the sample is not disturbed as in prior collection devices.

Exemplary embodiments of a collection device and system in accordance with the present invention may be used with a catheter, needle, or other body-indwelling device for collecting fluids, such as blood, from the body. Exemplary devices includes a housing, a piston rod movably mounted within the housing, and a control mechanism which controls the movement of the piston with respect to a fluid containing syringe cartridge removably positioned within the housing. The piston rod includes a plunger and a shaft in communication with the collection mechanism. The plunger and collection mechanism are connected by a holder. The system includes one or more latches to hold the device in an extended position until the device is to collect the body fluid sample.

The sample is collected by attaching the device to a catheter, needle, vacuum-filled vial, or the like, and releasing the latch(es) once body fluid has filled and is in communication with the device. The latches when released allow the plunger be moved inside the housing by a spring and the friction between the walls of the housing and the plunger is such that the friction provides a resistance force proportional to the spring force driving the plunger cylinder, so that a constant speed of the plunger is attained by the system. This provides a low shear force in the fluid being collected, which contributes to lower degradation of the sample, so that the fluid sample can be used directly in the testing system. The low shear force extraction also has a second benefit for people with fragile veins because it extracts at a slow and constant rate to avoid collapsing their veins. The system can alternatively be used with a needle or other hollow cannula if the patient does not want to use a catheter. Additionally, provision is made to expel the collected fluid from the system.

Referring to the drawing figures, like reference numerals designate identical or corresponding elements throughout the several figures.

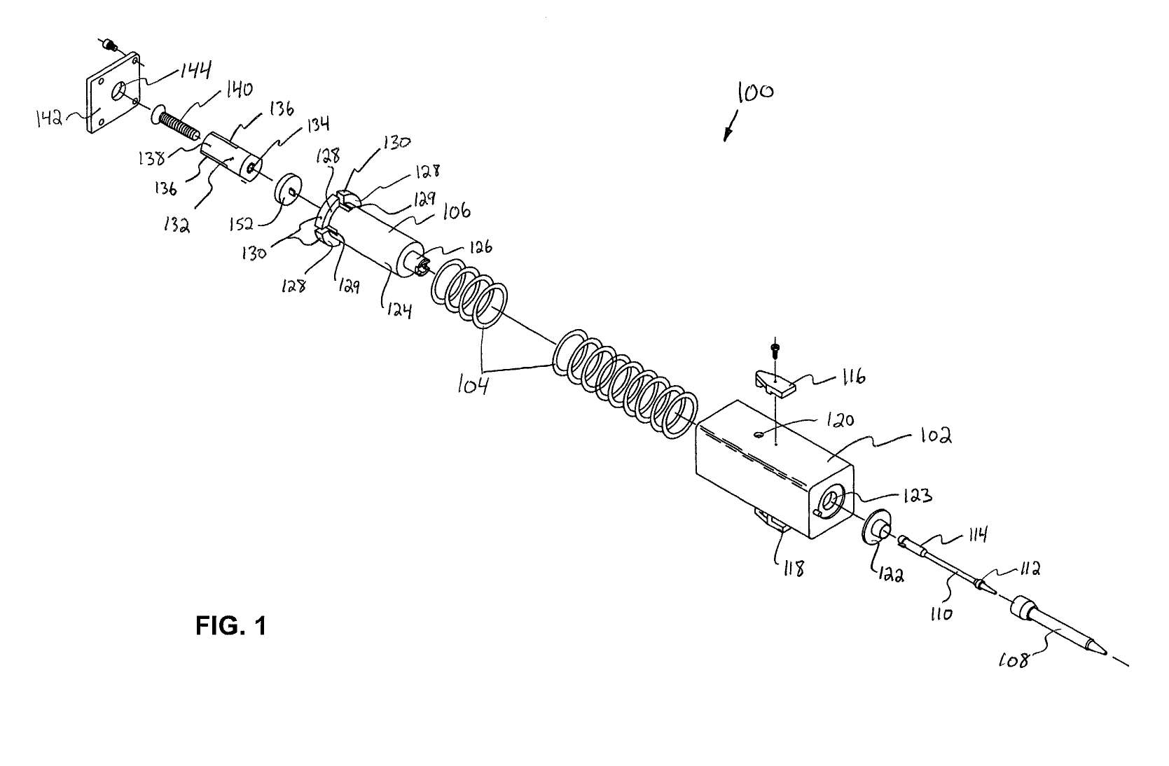

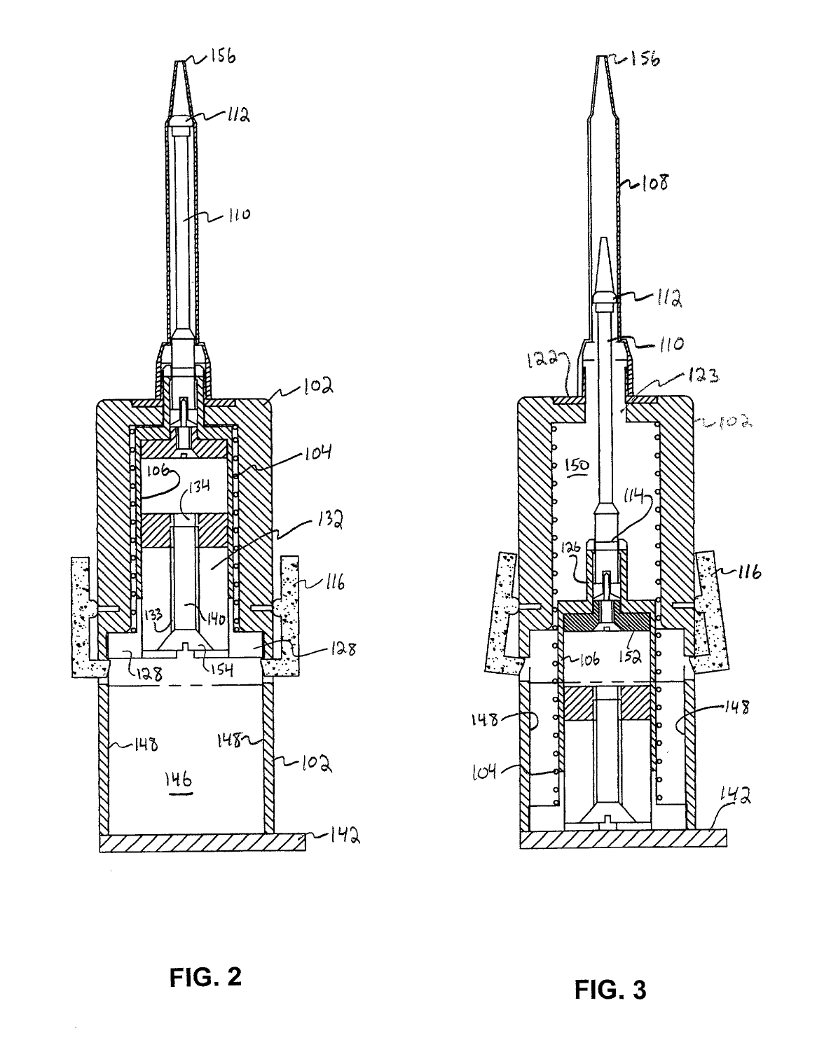

FIG. 1 illustrates an exemplary embodiment of an apparatus in accordance with the present invention. Fluid collection and dispensing device 100 includes an elongate and hollow housing 102, a spring 104, a cylindrical and hollow plunger 106, and a distal cannula 108. A piston 110 is slidably received in the hollow interior of the cannula 108, and includes a distal seal 112 which engages with the interior wall of the cannula (see FIGS. 2 and 3) sufficiently to pull a vacuum when retracted proximally. The piston includes a proximal end 114 which is configured to attach to a coupling inside the housing, described in greater detail below.

The housing 102 includes at least one movable or pivotable latch 116, and preferably a second latch 118, disposed on opposite sides of the exterior of the housing. The housing itself includes a hole 120 which leads into the interior of the housing through which portions of the latch extend and engage the plunger 106 (see, again, FIGS. 2 and 3), and from which these same portions can be moved to permit the plunger to move. A flanged coupling 122 is mounted around a distal opening 123 in the housing, and provides a mount for the proximal end of the cannula 108. According to yet another aspect of the present invention, the plunger 106 can be made longer so that the user of the device 100 can pull on the plunger to release it.

The plunger 106 includes a cylindrical barrel 124 from which a distal cylindrical extension piece 126 extends. Piece 126 is sized and shaped to receive therein or thereon the proximal end 114 of the piston 110. When the plunger 106 is in a proximal, retracted orientation, the piston 110 extends through the coupling 122, distal opening 123, spring 104, and into a first hollow interior space 150 (see FIG. 3) of the housing 102. The plunger 106 includes at least one, preferably at least two, outwardly extending spring tabs 128 which are separated by slots 129 through the barrel 124. Each tab 128 includes an outer friction surface 130 which is oriented to bear against an inner surface of the housing 102, described in greater detail below. As discussed generally above, the friction force generated between surface(s) 130 and the inner surface of the housing 102 is used to counterbalance the spring force generated by the spring 104 to result in a substantially constant velocity of the piston 110 relative to the cannula 108.

A split nut 132 having an at least partially threaded interior bore 133 (see FIG. 2) is permanently mounted in the hollow interior of the plunger 106. A distal interior threaded portion 134 receives the distal end of a screw 140, which extends proximally. At least two, and preferably a plurality of proximal slots 136 extend through the nut 132 and define at least one, and preferably a plurality of outwardly deflectable fingers 138. The screw 140 acts as a speed adjustment device as described in greater detail below.

A coupling disc 152 is positioned in the interior of the plunger 106 and is secured, either permanently or releasably, to the proximal end 114 of the piston 110, and thus secures the piston to the plunger so that the two elements move longitudinally together. A proximal end plate 142 including an access hole 144 is secured to the proximal end of the housing. The access hole is sized to permit an appropriately shaped driving tool (not illustrated), such as a screwdriver or the like, to be inserted through the plate 142 to turn the screw 140.

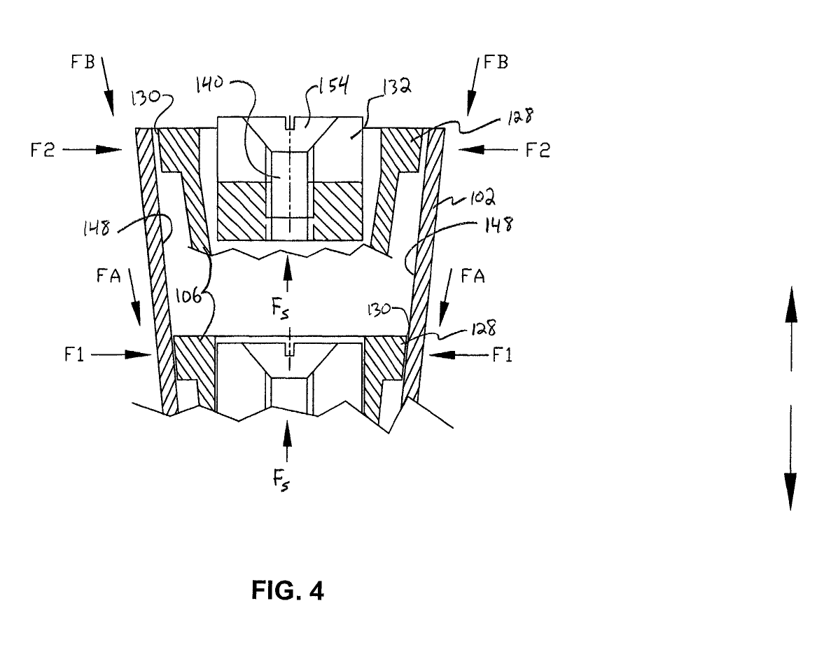

FIG. 4 illustrates the balancing of forces to result in a substantially constant piston-and-plunger retraction velocity. In FIG. 4, two exemplary positions of the split nut 132 relative to the plunger 106 are illustrated, an upper and a lower example. In FIG. 4, the tapers of several of the elements have been exaggerated to be easier to visualize, and one of ordinary skill in the art will appreciate that a much less severe taper is also within the scope of the present invention. In FIG. 4:

F_{A}=Force of friction, lower example

F_{B}=Force of friction, upper example

F₁=Normal force of wall 148 against tabs 128, lower example

F₂=Normal force of wall 148 against tabs 128, upper example

F_{S}=Spring force from spring 104

In the upper example, the screw 140 is relatively proximally located, so that the screw head 154 does not bear against the proximal portions of the split nut 132. Thus, the deflectable fingers 138 do not bear on, or bear less on, the inner surface(s) of the plunger 106. Thus, there is less normal force F₂ generated between the surface(s) 132 and the inner surface 148 of the housing, and therefore there is less frictional force to counteract the spring force F_{S}.

The lower example illustrates when the speed adjustment screw 140 is adjusted to push the deflectable fingers 138 outward. The fingers 138 thus bear, or bear more than in the upper example, on the inner surface of the plunger 106, which in turn generates a greater normal force F₁ between the surface(s) 130 and the surface 148. This generates a greater frictional force F_{A} to counteract the spring force F_{S}. By thus simply turning the screw 140, the device 100 can be adjusted to achieve a nearly constant piston velocity. Additionally advantageous, as the surfaces 130 and 148 begin to wear with use, the device 100 can be readjusted to achieve constant velocity piston motion.

Turning now to FIGS. 2 and 3, in which longitudinal cross-sectional views of the assembled device 100 are illustrated, the operation of the device will now be described with reference to the drawing figures. In FIG. 2, the device 100 is in an extended position with the piston 110 positioned distally in the cannula 108 and the plunger 106 positioned in the distal portions of cavity 150. The tabs 128 are engaged and held by the latches 116, 118, through the holes 120. The spring 104 is thus compressed between the tabs 128 and the distal interior wall of the housing 102. In this orientation, the device 100 is ready to take a fluid sample.

When it is desired to gather a fluid sample, the distal tip 156 of the cannula 108 is placed in fluid communication with the fluid, such as by inserting it into the hub of a catheter or needle, or into a drop of fluid from a puncture. The latches 116, 118 are then released, which releases the plunger 106. The spring 104 is therefore unrestrained, and pushes the plunger 106 proximally. The friction surfaces 130 of the tabs 128 bear against the outwardly tapered interior wall 148 of the housing 102, and generate a frictional force therewith that counteracts the spring force and results is the plunger moving proximally at a substantially constant speed. As will be readily appreciated by one of ordinary skill in the art, the interior wall 148 of the housing 102 is tapered outwardly from the distal end of the cavity 146 to the proximal end, so that the friction force tapers off at substantially the same rate that the spring force diminishes. Thus, the plunger is made to move proximally at a substantially constant speed over the length of its travel in the housing 102, with the tabs 128 and housing 102 together cooperating as a brake mechanism.

When the plunger reaches the end plate 142, it stops its proximal motion, and the piston 110 has been withdrawn relative to the cannula 108, drawing a fluid sample into the interior of the cannula distal of the seal 112. When it is desired to dispense the fluid thus collected, the plunger 106 is pushed distally, as by inserting a pushrod or the like (not illustrated) through the hole 144 and pushing the plunger.

According to other aspects of the present invention, the coil spring 104 can be alternatively replaced with an air spring, for which appropriate seals are provided between the outer surface of the piston 110 and the inner surface of the housing 102, such as at the distal opening 123, and between the outer surface of the plunger 106 and the inner surface of the housing 102 in the region of cavity 150.

While the invention has been described in detail with reference to preferred embodiments thereof, it will be apparent to one skilled in the art that various changes can be made, and equivalents employed, without departing from the scope of the invention.

What is claimed is:

1. A fluid sample collection device comprising:
a housing having a hollow interior;
a plunger positioned at least partially within said housing interior and movable within said housing;
a piston attached to the plunger and extending from the housing;
a spring positioned to move the plunger in a first direction;
a wall surrounding and engaging a portion of the plunger to generate a friction force when the plunger moves relative to the wall; and
a brake mechanism in the plunger which adjusts the friction force.

2. A fluid sample collection device in accordance with claim 1, wherein the housing has a proximal end and a distal end, the wall is a part of the housing, and the wall tapers outwardly proximally.

3. A fluid sample collection device in accordance with claim 1, wherein the plunger includes an outwardly extending flexible tab, the tab including an outer surface which engages with the wall to generate the friction force.

4. A fluid sample collection device in accordance with claim 3, the plunger includes a plurality of outwardly extending flexible tabs, each tab including an outer surface which engages with the wall to generate the friction force.

5. A fluid sample collection device in accordance with claim 1, further comprising a hollow cannula mounted to the exterior of the housing, the piston at least partially extending into the cannula.

6. A fluid sample collection device in accordance with claim 1, wherein the spring is a coil spring.

7. A fluid sample collection device in accordance with claim 1, wherein the spring is an air spring.

8. A fluid sample collection device in accordance with claim 1, wherein the brake mechanism includes a split nut mounted in the plunger, and a screw received in the split nut, movement of the screw relative the split nut moving a portion of the split nut outwardly to press against the inner surface of the plunger.
